(19) United States
(12) Patent Application Publication
(10) Pub. No.: US 2025/0380966 A1
(43) Pub. Date: Dec. 18, 2025

(54) METHODS AND APPARATUS FOR IMMOBILIZING AN EXTERIORIZED UTERUS

(71) Applicant: Shahram Daneshmand, La Jolla, CA (US)

(72) Inventor: Shahram Daneshmand, La Jolla, CA (US)

(21) Appl. No.: 19/239,931

(22) Filed: Jun. 16, 2025

Related U.S. Application Data
(60) Provisional application No. 63/660,688, filed on Jun. 17, 2024.

(51) Int. Cl. A61B 17/42

(52) U.S. Cl. CPC A61B 17/4241; A61B 2017/4216

(57) ABSTRACT

Methods and apparatus for immobilizing an exteriorized organ. In one specific implementation, an immobilizing device may be used to secure an exteriorized uterus for post-delivery suture (instead of requiring a physician or their assistant to hold the organ for suture). In addition to reducing fatigue, the exemplary techniques described herein may have a host of other benefits. By relieving physicians, physician assistants, midwives, or family medicine providers, hospitals can redirect these professionals to attend to other patients, thereby optimizing patient care and increasing revenue streams.

### PRIORITY

This application claims the benefit of priority to U.S. Provisional Patent Application Ser. No. 63/660,688 filed Jun. 17, 2024, and entitled “METHODS AND APPARATUS FOR IMMOBILIZING AN EXTERIORIZED UTERUS”, which is incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records but otherwise reserves all copyright rights whatsoever.

### TECHNICAL FIELD

This disclosure relates generally to the field of surgical implements. More particularly, the present disclosure relates to methods and apparatus for post-delivery management of the uterus during cesarean sections.

### DESCRIPTION OF RELATED TECHNOLOGY

The current landscape of obstetric care faces several challenges, including a shortage of labor, closure of labor and delivery units across the country, high costs associated with assistance during cesarean sections, assistant fatigue, and consequent loss of revenue. Addressing these unmet needs requires innovative solutions that streamline procedures, reduce costs, and improve efficiency.

### BRIEF DESCRIPTION OF THE DRAWINGS

FIG. 1 is a graphical representation of a human uterus.

FIG. 2 is a graphical representation of a vacuum assisted delivery device and a surgical clamp.

FIG. 3 depicts a human uterus that was immobilized with a vacuum assisted delivery device and surgical clamps in an improvised manner during a cesarean section procedure.

FIG. 4 depicts a generalized apparatus for immobilizing an exteriorized human organ.

### DETAILED DESCRIPTION

In the following detailed description, reference is made to the accompanying drawings. It is to be understood that other embodiments may be utilized, and structural or logical changes may be made without departing from the scope of the present disclosure. Therefore, the following detailed description is not to be taken in a limiting sense, and the scope of embodiments is defined by the appended claims and their equivalents.

Aspects of the disclosure are disclosed in the accompanying description. Alternate embodiments of the present disclosure and their equivalents may be devised without departing from the spirit or scope of the present disclosure. It should be noted that any discussion regarding “one embodiment”, “an embodiment”, “an exemplary embodiment”, and the like indicate that the embodiment described may include a particular feature, structure, or characteristic, and that such feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the features, structures, or characteristics of the given embodiments may be utilized in connection or combination with those of any other embodiment discussed herein.

Various operations may be described as multiple discrete actions or operations in turn, in a manner that is most helpful in understanding the claimed subject matter. However, the order of description should not be construed as to imply that these operations are necessarily order dependent. The described operations may be performed in a different order than the described embodiments. Various additional operations may be performed and/or described operations may be omitted in additional embodiments.

### 1 The Cesarean Section

A cesarean section (C-section) is a surgical procedure used to deliver a baby through incisions in the abdomen and uterus. It is often performed when a vaginal delivery would put the baby or mother at risk. A first incision may use a horizontal incision just above the pubic hairline (or a vertical incision from just below the navel to just above the pubic bone) to expose the abdominal wall. The abdominal wall is opened by carefully separating/cutting the layers of tissue and muscle to expose the uterus. A uterine incision is then made, usually horizontally across the lower segment. The human baby and placenta may then be delivered through the incisions.

Uterine exteriorization is a technique used during some cesarean sections. Specifically, once the baby is delivered and the placenta is removed, the emptied uterus may be gently brought out of the abdominal cavity. The uterine incision is sutured while the uterus is exteriorized. After suturing, the uterus is carefully placed back into the abdominal cavity, and the remaining layers of the abdominal wall are closed.

Uterine exteriorization provides better access and visibility for the surgeon to suture the uterine incision. This can provide better (more accurate) stitching, smaller stitches, and a faster more efficient process. These benefits may directly translate to reduced blood loss, faster recovery and improved healing.

While uterine exteriorization is a common practice, it is not universally performed since the exteriorized uterus must be held in place (immobilized) during the suturing process. Traditionally, a surgical assistant is crucial for maintaining uterine retraction (if the uterus is exteriorized), facilitating suturing, and managing blood within the operative field during cesarean sections for improved exposure. However, an additional person in an already crowded operative field may increase the chance of accidents (e.g., accidental needle sticks), constrain personnel schedules, and/or increase the cost of labor.

### 2 Cesarean Section Assistant Device

Exemplary embodiments of the present disclosure are directed to a cesarean section assistant device and its methods of use. The cesarean section assistant device immobilizes the exteriorized uterus during cesarean sections. This allows the surgeon/surgical assistants to allocate their efforts more efficiently and mitigate fatigue, particularly during prolonged procedures. In addition to improved surgical outcomes, the exemplary techniques may also reduce labor requirements and costs of the procedure.

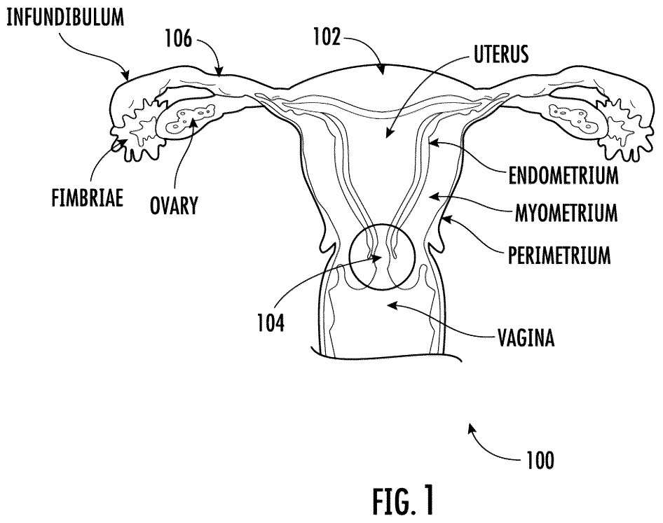

FIG. 1 is a graphical representation of a human uterus 100. Of particular note, the uterine fundus 102 is the top part of the uterus, opposite the cervix 104. It is a broad, curved, upper area of the uterus and is located above the openings of the fallopian tubes 106.

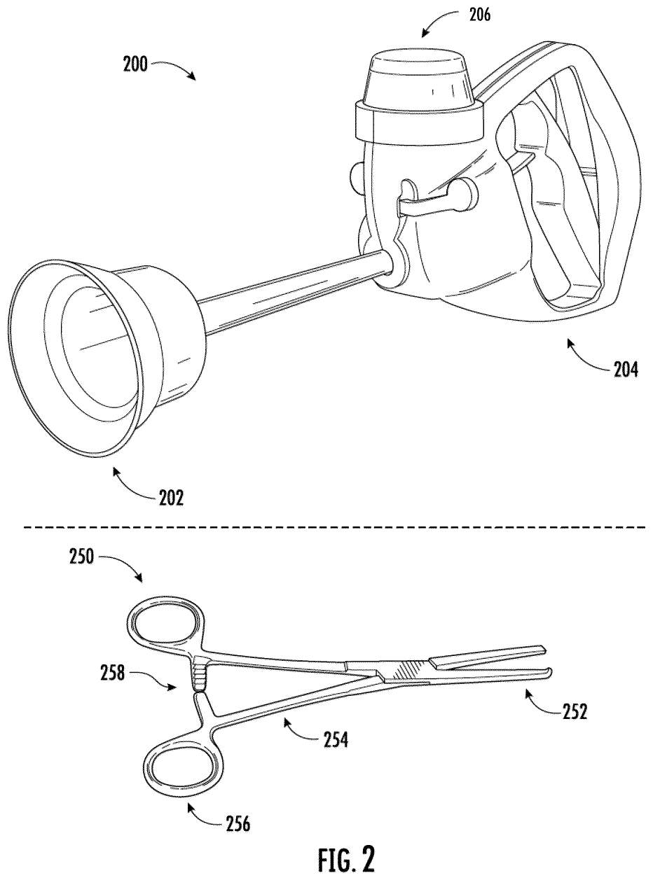

FIG. 2 is a graphical representation of a vacuum assisted delivery device 200 (also referred to as a “ventouse”) and a surgical clamp 250. The vacuum assisted delivery device 200 includes a bell suction cup 202 and a manually operated suction pump 204 that creates a vacuum within a chamber (not shown, interior of device). The vacuum assisted delivery device 200 may additionally have a pressure gauge 206. Here, the vacuum assisted delivery device incorporates a Bell suction cup with a 64 mm diameter and features an extended shaft linked to a vacuum chamber.

The surgical clamp 250 includes a scissors-like set of jaws 252 (that may or may not have teeth) and are configured to grasp or hold material. The shanks 254 may include finger rings 256 to provide manual control of closure and a ratchet 258 to lock the jaws at various degrees of tightness.

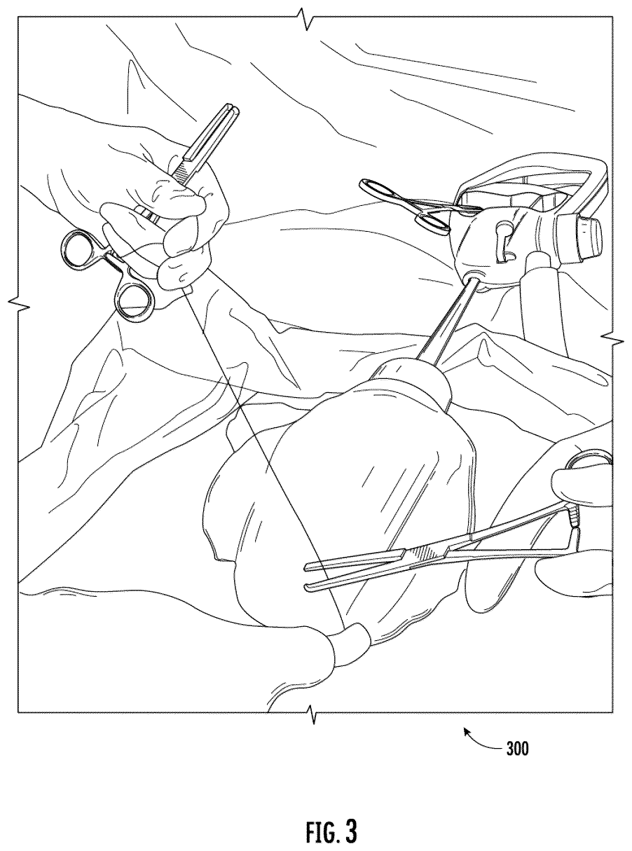

FIG. 3 depicts a human uterus that was immobilized with a vacuum assisted delivery device and surgical clamps in an improvised manner during a cesarean section procedure. After delivery of the newborn and placenta, the uterus was removed and the bell-shaped cup was positioned onto the uterine fundus. Utilizing the manually operated suction pump, suction was applied to the fundus via the vacuum assisted delivery device. The vacuum assisted delivery device was then secured with surgical clamps to a surgical drape. In this manner, the uterus was immobilized outside the abdomen without requiring any further handling (“hands-free”). The depicted surgical assistant then e.g., could use one hand for suture following and the other for managing the visual field, etc. Upon closure of the uterine incision, the trigger vacuum release mechanism releases the vacuum assisted delivery device from the uterus, which could then be re-inserted into the patient's body.

Artisans of ordinary skill in the related arts will readily appreciate that the improvised apparatus and techniques described above demonstrate the feasibility and benefits of the techniques described herein. As a practical matter, however, the improvised device was constructed with a vacuum assisted delivery device designed for delivering a baby. The bell-shaped cup is configured to attach to the crown of a fetus (rather than the uterine fundus) and provide substantial suction to assist in delivery. The vacuum assisted delivery device also has an extended shaft to allow placement and manipulation within the birth canal, and handles to allow the surgeon to exert significant pulling force, etc. More directly, a device that is specifically designed to immobilize the exteriorized uterus during suturing may be constructed to more gently attach to the uterine fundus and provide greater stability during operation. Such a device may also be manufactured at reduced cost and form factors that are more suitable for this procedure within the crowded operating room.

In addition to reducing fatigue, the exemplary techniques described herein may have a host of other benefits. For example, needle injuries during cesarean sections are especially prevalent among training residents and pose significant risks. The improved stability of the exemplary technique reduces such injuries by providing stable uterine retraction. This not only safeguards healthcare professionals but also minimizes associated costs and potential viral infections, thereby enhancing patient safety and reducing healthcare expenditures. Furthermore, the exemplary technique may offer flexibility in resource allocation (especially for institutions where a physician's presence is not mandatory during the post-delivery suturing process). By relieving physicians, physician assistants, midwives, or family medicine providers earlier in the procedure, hospitals can redirect these professionals to attend to other patients, thereby optimizing patient care and increasing revenue streams. In addition, such techniques may be a cost-effective alternative to employing a physician assistant for cesarean section assistance. By reducing the need for additional personnel and streamlining procedural support, the Cesarean Section Assistant Device not only lowers operational costs but also enhances the cost-effectiveness of healthcare delivery, aligning with the goals of health plan reimbursement models.

### 3 Exemplary Methods and Apparatus

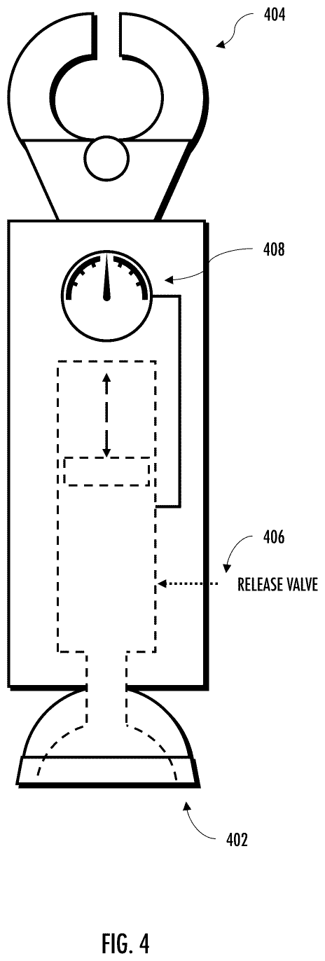

In one aspect, an apparatus for immobilizing an exteriorized uterus is disclosed. FIG. 4 depicts a generalized apparatus for immobilizing an exteriorized human organ. The apparatus may include: an attachment mechanism 402 and a mounting mechanism 404. Some variants may additionally include one or more of e.g., a release mechanism 406, monitoring apparatus 408, and/or automated actuator control logic (not shown).

Functionally, the attachment mechanism 402 attaches the apparatus to a portion of anatomy (e.g., an organ that is exteriorized). In one embodiment, the attachment mechanism is configured to attach to a fundus of the uterus. Other embodiments may attach to other portions of the uterus e.g., cervix, corpus, etc. While the foregoing discussion is presented in the context of cesarean procedure, the concepts may be applied to other types of surgical procedures that externalize anatomical organs. Such procedures may include e.g., laparotomy, nephrectomy, colostomy/ileostomy, as well as any organ transplant (liver, heart, lungs, etc.).

In one specific implementation, the suction cup is attached to a vacuum pump capable of creating suction pressure up to 0.6-0.8 kg/cm², which is equivalent to 60-80 kPa (kilopascals), or about 450-600 mmHg (millimeters of mercury). For reference, suction pressures below 200 mmHg (˜0.27 kg/cm²) are generally considered low suction, and unlikely to create bruising or contusions for most body tissues. 200-400 mmHg (˜0.27-0.55 kg/cm²) is considered moderate suction pressure which may create bruising when applied for significant duration (e.g., 10-20 minutes which would be a typical duration for the suturing procedure). Suction above 400 mmHg is likely to create bruises and may even cause deeper tissue damage if prolonged-nonetheless, there may be exigent circumstances that demand such high pressures.

In one embodiment, the suction cup may be removably and/or interchangeably attached to a vacuum pump. The suction cup may be coupled using a threaded fit, friction fit, pressure fit, magnetic fit, and/or any other coupling mechanism. In some variants, the suction cup may include a flexible extended shaft; in other variants, the suction cup may directly and/or rigidly connect to the vacuum pump.

Different suction cups may be used to accommodate differences in size of the exteriorized organ. For example, the transverse diameter of the uterine fundus typically ranges between 15-30 centimeters (cm), but atypical patients and/or conditions may span larger ranges. Differently sized suction cups and/or multiple suction cups may allow for a most robust attachment to accommodate for different patient needs.

Conceptually, one suction cup may immobilize the exteriorized organ in at least one dimension, relative to the mounting mechanism and anchor (described elsewhere); however, artisans of ordinary skill in the related arts will readily appreciate that additional suction cups may be used to further limit movement across more degrees of freedom. For example, two suction cups may immobilize the exteriorized organ in two dimensions, three suction cups may be used to immobilize the exteriorized organ in three dimensions, etc. Notably, the exteriorized uterus is connected to the human body by other structures (e.g., cervix, blood vessels, and other connective tissues, etc.), which may provide limited support as well. In other words, the degree of immobilization that is provided by the attachment mechanism 402 may be augmented by the exteriorized organ's own connective tissues and structures.

Furthermore, different suction cups may be used to accommodate material sensitivities (e.g., allergies, etc.). In one implementation, the suction cup may be manufactured from rubber, latex, plastic, silicone, metal, or other material. Medical applications have a wide variety of different materials which may be used to accommodate patients with immune disorders and/or allergic sensitivities. Common examples may include e.g., Polyvinyl Chloride (PVC), Polyurethane (PU), Polytetrafluoroethylene (PTFE/Teflon), and/or other polymers; Stainless Steel, Titanium, and/or other metals, etc. In some cases, materials may be coated, layered, or otherwise passivated with other chemicals to reduce the risk of contamination, improve sanitation, prevent undesirable complications, and/or accommodate patient conditions.

A suction cup works by creating a partial vacuum between the cup and the surface it's applied to (e.g., the exteriorized organ). This generates a pressure differential between the inside of the cup (lower pressure) and the surrounding atmosphere (higher pressure), causing the higher external air pressure to push the cup against the surface, holding it in place. In other words, the suction cup creates a pressure difference (“negative pressure” or “suction pressure”) between the sealed internal air pressure and the ambient air pressure.

Suction is strongly affected by the seal integrity e.g.; the sealing edge should prevent air from releasing the partial vacuum. While a bell-shaped suction cup can generate an adequate vacuum, other cup shapes may result in better sealing. In some cases, the suction cup may be shaped to more closely fit complement the shape of the exteriorized organ. In other cases, the suction cup may have more edges (e.g., accordion edges, etc.) that flexibly conform to the organ. This may be especially useful to ensure a good seal throughout the entire procedure.

Suction force is a function of the pressure difference and the area. For example, a suction pressure of 0.27 kg/cm² for ˜32 cm² (the effective surface area of a 64 mm bell suction cup) could sustain approximately 9 kg of force, which adequately secures a 1 kg uterus. More generally, increasing the surface area and/or the number of suction cups may allow e.g., better fit, gentle suction, etc. For example, a multi-cup attachment may allow smaller cups to be used along more points of the uterus. A larger cup may enable the same suction force with a lower pressure (reducing the likelihood of bruising, etc.). Various other combinations may be substituted with equal success.

In one embodiment, a vacuum pump removes gas from the sealed volume (between the suction cup and the exteriorized organ) to create the negative pressure. The vacuum pumps may additionally include gauges and/or safety release valves to assist with pressure control and safe operation. For example, the safety valve may prevent negative pressure from exceeding pressures that might permanently damage human tissue (e.g., above 600 mmHg of pressure). In some cases, safety release pressure may be configurable to accommodate patient needs. For example, natural variations in patient anatomy suggest that some uteruses may be able to sustain higher pressures than others; some patients may also have other complications that might limit suction pressures (e.g., trauma, etc.).

While the improvised device uses a hand-pumped vacuum pump to generate negative pressure, other implementations may be substituted with equal success. Simple suction cups may not even have a vacuum pump—e.g., the clinician may press the suction cup to the fundus to create a seal and expel air (the negative pressure is created as the suction cup attempts to return to its unpressed shape). Mechanically assisted suction cups may use a lever or twisting mechanism to create suction pressure, etc.

So-called “smart” systems may use actuated pumps and/or electronics to maintain suction pressure to ensure robust attachment throughout the suturing procedure. Notably, a typical uterus weighs 1000-1200 grams at delivery, a significant portion of which is blood. The typical uterus loses 500-1000 milliliters (ml) of blood during a cesarean-which can cause significant changes in size and shape. In other words, automated pumps may sense pressure loss (attributed to movement and/or shrinkage/drainage from fluid loss, etc.) and dynamically compensate as needed.

More capable systems may also monitor the health of the exteriorized organ during the procedure. Exteriorized organs are still connected to the patient, and their blood supply may be inadvertently pinched or otherwise impinged during the procedure. Examples of vital signs that may be monitored may include e.g., heart rate, blood pressure, oxygen saturation, radiotracers/radiopharmaceuticals, as well as metabolites (e.g., fluids, medicines, anesthesia, etc.) and/or their rate of metabolism.

In addition to automation (dynamic assistance, etc.), smart systems may also offer a variety of different user interfaces to assist the surgeon during operation. Visual interfaces (graphical user interfaces (GUIs)) may be used to display dense layers of information in a variety of intuitive ways (e.g., real-time monitoring, historical data, etc.). Capacitive touchscreen GUIs may also be readily sanitized (which may be an issue for user input devices e.g., a keyboard, mouse, etc.). During an operation, the surgeon may not be able to look at a GUI, however, and alternative user interfaces may be preferred e.g., voice commands, audible alerts, and/or haptic (rumble box) style interfaces. For example, a surgeon may be able to vocally command suction to increase, decrease, release, etc. without averting their gaze and/or actively using their hands.

As a brief aside, suction-based attachment can be used to immobilize an exteriorized organ without compression. While it is possible to “squeeze” the exteriorized uterus to hold it in place (e.g., using clamps, etc.), compression-based immobilization may impact blood flow—this can also be compounded by the fact that the organ's own weight is unsupported when it is exteriorized (since its supporting connective tissues may be severed or temporarily re-positioned, etc.), and it may pinch-off its own blood supply. Suction is not without its own challenges since it is possible to create contusions with high suction (ruptured blood vessels that causes blood to leak into surrounding tissues); nonetheless, low amounts of suction can be used over large surface areas to generate enough tensioning force to immobilize the exteriorized organ without impacting normal blood flow.

More generally, artisans of ordinary skill in the related arts will readily appreciate that a variety of other attachment mechanisms and/or optimizations may be substituted with equal success, given the contents of the present disclosure. For example, other implementations may use mechanical and/or chemical fastening means to attach to the exteriorized organ. Mechanical examples may include e.g., straps, clips, clamps, rings, belts, and/or any other physical fastening mechanism. Chemical examples may include e.g. adhesives, glues, epoxies, and/or other adhesive substances.

As but one such example, the attachment mechanism 402 may be a clamp that uses one or more appendages to grasp the externalized organ. A uterine clamp may comprise two plates to “sandwich” the uterus (immobilizing the uterus via friction). The clamping plates may be rectangular, circular, or other shape. The plates may have a surface that is planar, concave, or convex. Plate dimensions may be selected to physically complement the patient uterus (e.g., 10 cm wide 15 cm long for a typical uterus, etc.). Plates may be rigid or flexible, and constructed of various materials as discussed above.

Manually operated variants may use springs and/or other tensioning devices to ensure that the plates have sufficient pressure to immobilize the uterus. In some such variants, the plates may further lock into position, etc. For example, a ratcheted lock mechanism may allow the jaws to ratchet into position, a step at a time. The surgeon can ratchet together the jaws, as-needed. This maintains a firm grasp on the uterus without requiring the surgeon to continue squeezing the grips and/or to permit the clamp to be used to manipulate the uterus into a variety of positions during the postpartum phase of delivery while the uterus is held securely between the plates. Smart variants may use pneumatics, or other actuation to physically control the amount of grasping pressure.

As another such example, the attachment mechanism 402 may use an inflatable cuff which can be wrapped around the uterus and inflated to a desired pressure (immobilizing the uterus via friction). A uterine cuff may comprise one or more air bladders (pneumatic) or liquid bladders (hydraulic) which can be used to flexibly conform to the physical contours of the exteriorized uterus. Manually operated variants may use air/liquid pumps to fill the bladders. Gauges may be used to inform the clinician as to the amount of inflation; this may allow the clinician to adjust pressure by sight. As discussed elsewhere, smart variants may automatically monitor and dynamically manage inflation pressure.

Still other variations of the foregoing will be readily appreciated by artisans of ordinary skill in the related arts, given the contents of the present disclosure.

Referring back to FIG. 4, the mounting mechanism 404 mounts the apparatus to an anchor, limiting motion of the apparatus in at least one degree of movement. A degree of movement refers to the extent or range of motion allowed or available in a particular direction or plane of movement, especially in the context of the human body. In some cases, multiple degrees of movement may be restricted. Here, one degree of freedom would refer to movement in a single plane, two degrees of freedom may refer to movement in two planes, three degrees of freedom may refer to movement in three planes.

Examples of degrees of movement may include: flexion and extension, abduction and adduction, rotation, circumduction, elevation and depression, protraction and retraction. Here, flexion refers to motion that decreases an angle between two body parts (e.g., the exteriorized organ and the rest of the body), extension refers to motion that increases an angle between two body parts. Abduction refers to motion that moves a body part away from a midline whereas adduction refers to motion that moves a body part toward the midline. Elevation and depression refer to motion that moves a body part up/down. Protraction and retraction refer to motion that moves a body part forward/backward. Various other degrees of movement may be constrained, the foregoing being purely illustrative.

Suitable anchors may be located in a fixed position relative to the patient. In some cases, the anchor affixed to the patient or their supporting equipment; e.g., the anchor may be a bed, operating table, arm board, leg stirrup, head rest, etc. In other cases, the anchor is affixed to a fixed structure of the operating room; e.g., overhead lights, machinery (e.g., patient monitors, anesthesia machine, electrosurgical unit, etc.), and/or other room fixtures. Some anchors may be movable/mobile and then locked into place (e.g., crash carts, IV poles, etc.). Artisans of ordinary skill will readily understand that the mounting mechanism 404 may mount to any number of anchors.

In one embodiment, the mounting mechanism 404 includes one or more clamps. A clamp applies grip pressure to an object or between objects to increase friction; with sufficient friction, the clamp prevents movement-lower amounts of pressure may be used to slow/impede motion (e.g., where complete immobilization is not necessary). Clamps generally include one or more jaws to grip the anchor, an adjustment mechanism to adjust the amount of grip pressure (friction), and a mechanism to tighten/release the clamp.

In some variants, the mounting mechanism 404 may rigidly “lock” into place/position. Locking mechanisms ensure that the jaws securely grip the anchor with a consistent grip pressure. Ratcheting and/or locking mechanisms also hold the anchor without needing continuous manual force (allowing for hands-free operation). Locking mechanisms also provide other safety benefits (e.g., resist vibration, prevent unexpected shifting, etc.). Locking mechanisms may also include quick-release and/or toggle-latching mechanisms to speedup workflow (e.g., fast clamping and release).

A variety of different clamp types may be substituted with equal success. Within the medical area, a variety of different clamps are used for a variety of different applications (e.g., Mosquito, Kelly, Crile, Pean, Allis, Babcock, Kocker, Bachhaus, Edna, Foerster, Satinsky, Bulldog, etc.). Other examples of clamps may include C-clamps, bar clamps, spring clamps, toggle clamps, etc.

Much like the attachment mechanism 402, the mounting mechanism 404 may also be removable and/or interchangeable. Similarly, multiple clamps and/or different clamps may be used to mount to different fixtures and/or anchor points. Different mounting mechanisms may be used to accommodate differences in the anchor points (e.g., mounting to a bed versus a crash cart, etc.). Mounting mechanisms may also be constructed of different materials to reduce the risk of contamination, improve sanitation, prevent undesirable complications, and/or accommodate patient conditions. Similarly, smart systems may electronically actuate and/or release the mounting mechanism 404, etc.

While the foregoing discussion is presented in the context of a mechanical clamp, artisans of ordinary skill will readily appreciate that any mounting mechanism may be substituted with equal success. Other implementations may use mechanical, chemical, and/or electromechanical fastening means. Mechanical examples may include e.g., clips, clamps, rings, belts, and/or any other physical fastening mechanism. Chemical examples may include e.g. adhesives, glues, epoxies, and/or other adhesive substances. Electromechanical examples may include e.g., magnets, electromagnets, etc.

Functionally, the release mechanism detaches the apparatus from the anchor and/or the portion of anatomy. Once detached from the exteriorized organ (or from the anchor), the exteriorized organ can be moved freely e.g., to be re-inserted into the patient, moved into a different position to facilitate a different suture location, etc.

Release mechanisms may vary depending on the nature and/or operation of the attachment mechanism and/or mounting mechanism. For example, a suction cup may be released by releasing negative pressure, a clip may be mechanically released, etc.

In one embodiment, the release mechanism may release the attachment mechanism 402 by allowing the sealed volume (between the suction cup and the exteriorized organ) to equalize with atmospheric pressure, thus releasing suction. More sophisticated manual variants may provide fine control (e.g., twist/button valves, etc.) which allow the user to reduce the negative pressure in a controlled manner. Smart variants may finely regulate suction pressure using sensors to sense pressure, and pumps to add/remove negative pressure as needed. Other release mechanisms may e.g., release pressure from a uterine cuff, unlock uterine clamps, etc.

In one embodiment, the release mechanism may release the mounting mechanism 404 by opening the jaws to release the anchor. Some variants may allow for fine control of the jaws (e.g., allowing a slow release of grip pressure). Other variants may allow for a quick-release (e.g., spring-loaded and/or cam-based tension may allow the spring/cam to return to its un-tensioned state, etc.). Toggle-latching/toggle-releases may use a pivoting linkage that has a “toggle point” (a change in mechanical advantage) which allows for a secure latch-up and spring-like quick release.

In one aspect, a method for immobilizing an exteriorized organ is disclosed. The method may include removing the organ from a body cavity, attaching an apparatus to the organ, and mounting the apparatus to an anchor. Once the surgical procedure has been completed, the organ may be detached from the apparatus and re-inserted into the body cavity.

### 4 Other Considerations

While exteriorizing organs can offer benefits in terms of access and visibility during surgery, the potential risks and disadvantages often lead surgeons to prefer alternative techniques when possible. Exteriorization introduces multiple complexities to the surgical procedure e.g., increased risks of infection, potential for organ damage during manipulation/exteriorization, increased anesthesia, hemodynamic instability, and/or potentially longer post-operative recovery. Also, the heightened concern and increased risks of exteriorization are often addressed with increased size and/or skill of the surgical team at the outset of the procedure. Conventional wisdom typically suggests that the exteriorized organ should be closely monitored and handled; so, in many cases, a surgical assistant is specifically tasked with this during the procedure. In other words, surgical procedures that exteriorize organs are typically performed by highly skilled surgical teams and are only undertaken when the benefits outweigh the potential risks.

Within the context of obstetrics, vaginal childbirth is typically preferred at the outset-a cesarean section is used as a fallback when complications develop that could affect the health of the mother and/or child (ren). Furthermore, the uterus remains in the body for the cesarean procedure but is exteriorized, only when necessary for post-delivery suture. Importantly, the uterus is smaller (since it no longer contains the baby and placenta), and is more readily exteriorized, sutured, and re-inserted with substantially less trauma. In fact, unlike most other exteriorization procedures, closing the suture is not the primary focus, but is instead a post-delivery procedure (a physician's presence may not even be needed during the post-delivery suturing process). For these reasons, many of the conventional assumptions for exteriorization-based surgeries are less important. Here, a smaller team is desirable, suturing may be performed by surgical assistants, and the danger of trauma (while still significant) is reduced.

There are some uterine clamps that exist. For example, U.S. Pat. No. 9,168,061, filed Dec. 15, 2014, by DiBenedetto (hereinafter “DiBenedetto), describes a uterine clamp for treating postpartum hemorrhage and facilitating uterine repairs. Notably, DiBenedetto describes a medical clamp that includes a first and second curved plates that capture between them the major exterior walls of the uterus, and an operating handle having pivotally connected first and second actuating arms, each of which has a distal portion and a proximal portion with a grip for a user, whereby the distal portions are moved toward each other when the user moves the grips toward each other to capture the uterus between the plates. Ball-and-socket joints connect the distal handle portions of the actuating arms to respective plates. The ball-and socket joints enable rapid positioning of the clamp on the uterus to arrest emergency postpartum hemorrhaging, assist in exerting pressure on the uterus over the entire extent of the plates, facilitate manipulation of the uterus using the clamp, and permit other surgical interventions to be performed with the clamp in place. More directly, DiBenedetto is designed for a surgeon to actively compress to apply pressure and reduce hemorrhaging.

Similarly, vacuum cups have been used to exteriorize a uterus. For example, “A novel and effective technique to exteriorize larger uteri during laparotomic hysterectomy and myomectomy” to Lakhani (Wadia Journal of Women and Child Health. 1 (1), 2022) describes a procedure for “laparotomic hysterectomy” commonly referred to as “abdominal hysterectomy”. Specifically, the biggest challenge in such cases is to exteriorize the uterus from the incision prior to starting with the surgical steps. Lakhani describes the use of a silastic vacuum cup with a vacuum machine to facilitate the exteriorization of uterus from a regular Pfannenstiel incision. More directly, Lakhani describes a surgeon using significant suction force (600 mmHg) to pull a 20 week size gravid uterus from the body in preparation for surgery.

It will be appreciated that the various ones of the foregoing aspects of the present disclosure, or any parts or functions thereof, may be implemented using hardware, software, firmware, tangible, and non-transitory computer-readable or computer usable storage media having instructions stored thereon, or a combination thereof, and may be implemented in one or more computer systems.

It will be apparent to those skilled in the art that various modifications and variations can be made in the disclosed embodiments of the disclosed device and associated methods without departing from the spirit or scope of the disclosure. Thus, it is intended that the present disclosure covers the modifications and variations of the embodiments disclosed above provided that the modifications and variations come within the scope of any claims and their equivalents.

What is claimed is:

1. A method for immobilizing an exteriorized uterus, comprising:
exteriorizing a uterus from a patient after performing a cesarean section procedure;
attaching an immobilizing device to the uterus;
mounting the immobilizing device to an anchor;
closing a uterine incision of the uterus;
releasing the immobilizing device from the uterus; and
inserting the uterus into the patient.

2. The method of claim 1, where the immobilizing device comprises a suction cup, and where attaching the immobilizing device comprises applying a suction pressure to a fundus of the uterus.

3. The method of claim 2, where the suction pressure exerted by the suction cup is below 200 mmHg.

4. The method of claim 2, where an attachment force exerted by the suction pressure does not exceed 9 kg.

5. The method of claim 2, where releasing the immobilizing device from the fundus of the uterus comprises releasing the suction pressure.

6. The method of claim 2, where the suction pressure is dynamically adjusted to compensate for changes in a size or shape of the uterus.

7. The method of claim 1, where the immobilizing device comprises a clamp, and where mounting the immobilizing device comprises locking the clamp to the anchor.

8. An apparatus for immobilizing an exteriorized uterus, comprising:
an attachment mechanism configured to attach to a uterus;
a mounting mechanism configured to mount to an anchor; and
a release mechanism.

9. The apparatus of claim 8, where the attachment mechanism comprises at least one suction cup configured to attach to a fundus of the uterus.

10. The apparatus of claim 8, where the attachment mechanism comprises a mechanical clamp to grasp the uterus.

11. The apparatus of claim 8, where the attachment mechanism comprises an inflatable uterine cuff.

12. The apparatus of claim 8, where the mounting mechanism comprises a clamp.

13. The apparatus of claim 8, where the attachment mechanism is further configured to immobilize the uterus in at least one dimension, relative to the mounting mechanism.

14. The apparatus of claim 8, where the release mechanism is configured to release the attachment mechanism from the uterus.

15. A method for immobilizing an exteriorized organ, comprising:
exteriorizing an organ from a patient;
attaching an immobilizing device to the organ; and
mounting the immobilizing device to an anchor.

16. The method of claim 15, where the immobilizing device is configured to apply a suction pressure to the organ.

17. The method of claim 16, where the suction pressure is selected to minimize contusion.

18. The method of claim 17, where the suction pressure is below 200 mmHg.

19. The method of claim 15, where the exteriorized organ remains connected to the patient by at least one connective tissue and where the exteriorized organ is immobilized in at least one dimension relative to the anchor.

20. The method of claim 15, where the immobilizing device permits hands-free operation.
